(19) United States
(12) Patent Application Publication
(10) Pub. No.: US 2022/0332691 A1
(43) Pub. Date: Oct. 20, 2022

(54) Improved process for preparing epoxides from aldehydes or ketones

(71) Applicant: SALTIGO GmbH, Leverkusen (DE)

(72) Inventors: Andre Grossmann, Cologne (DE); Bjorn Schlummer, Bonn (DE)

(73) Assignee: SALTIGO GmbH, Leverkusen (DE)

(21) Appl. No.: 17/642,542

(22) PCT Filed: Sep. 9, 2020
(86) PCT No.: PCT/EP2020/075210

§ 371 Date: Mar. 11, 2022

(30) Foreign Application Priority Data
Sep. 12, 2019 (EP) 19196907.0

(51) Int. Cl. C07D 301/02; C07C 317/04

(52) U.S. Cl. CPC C07D 301/02; C07C 317/04

(57) ABSTRACT

The invention relates to an improved process for preparing epoxides from aldehydes or ketones by reacting a ketone or aldehyde with at least one sulfonium salt, and to sulfonium salts that may be used in the process of the invention.

The invention relates to an improved process for preparing epoxides from aldehydes or ketones by reacting a ketone or aldehyde with at least one sulfonium salt, and to sulfonium salts used in the process of the invention. Reactions of this type are also known by the name of the Corey-Chaykovsky reaction (L. Kurti, B. Czako, “Strategic Applications of Named Reactions in Organic Synthesis”). The epoxidation of the ketone or aldehyde proceeds via a sulfur ylide intermediate that can be generated from a sulfonium salt. The sulfonium salts used for this are normally generated from a dialkyl sulfide and an alkylating agent such as dimethyl sulfate or alkyl halides. The invention also relates to sulfonium salts that can be used in the process of the invention.

The epoxides prepared by the process according to the invention are for example intermediates or end products for various commercial uses, such as agrochemicals. The compound of formula (IV) is for example an intermediate for the azole fungicide cyproconazole (DE 3406993 A1). Also described therein is the preparation of 2-(4-chlorophenyl)-2-(1-cyclopropylethyl)oxirane from 1-(4-chlorophenyl)-2-cyclopropylpropan-1-one in the presence of dodecyldimethylsulfonium methyl sulfate and potassium hydroxide in a yield of 25% of theory.

EP 0205400 A2 describes for example the preparation of 2-propyloxirane from butyraldehyde in the presence of hexyldimethylsulfonium methyl sulfate and sodium hydroxide in yields of 70 to 80% of theory.

However, the processes described up to now often have the disadvantage that the dialkyl sulfides present in stoichiometric amounts at the end of the reaction (e.g. dodecyl methyl sulfide or hexyl methyl sulfide) can often be separated by distillation from the thermally less stable oxiranes only with difficulty or not at all. For this reason, it is usually not possible with the abovementioned processes on an industrial scale to obtain the oxiranes directly in high purity and/or in high isolated yield.

In contrast to these higher homologs, the simplest dialkyl sulfides such as dimethyl sulfide have on the other hand the disadvantage of exhibiting significantly lower reactivity in this type of reaction, which is associated with significant economic disadvantages.

There is thus still the need for, and hence the technical problem of providing, a variant of the Corey-Chaykovsky reaction that does not have the disadvantages of the prior art.

Surprisingly, a process for preparing epoxides has now been found, in which a ketone or aldehyde is reacted at least with a sulfonium salt of formula (I),

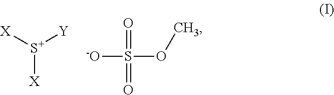

and/or a sulfonium salt of formula (II),

X₂SY⁺Z⁻ (II),

where in formula (I) and formula (II) X is methyl and Y is linear C₂ to C₁₁ alkyl, preferably ethyl, n-propyl, n-butyl, n-pentyl, n-heptyl, n-octyl, n-nonyl, n-decyl and n-undecyl,

and where in formula (II) Z is chloride, bromide, iodide or carbonate,

in the presence of a base, preferably alkali metal hydroxide, more preferably sodium hydroxide or potassium hydroxide.

Particular preference is given to the reaction of alkenes or ketones with a sulfonium salt of formula (I) in which X is methyl and Y is n-propyl and n-butyl.

The aldehydes or ketones used for the purposes of the invention are organic compounds having at least one carbon-oxygen double bond. The aldehydes or ketones may be linear, branched or cyclic. The aldehydes or ketones used in the process according to the invention may bear further substituents such as further aliphatic or aromatic radicals, which may in turn be substituted or unsubstituted.

In a preferred embodiment, the compound of formula (III) is used as the ketone

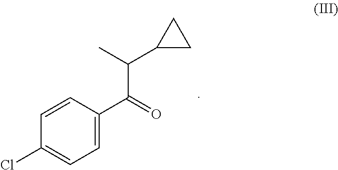

In the process according to the invention, this gives rise to the epoxide of formula (IV),

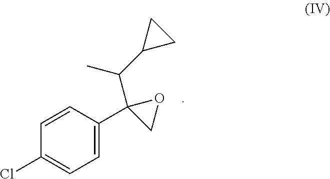

Particular preference is given to the reaction of the compound of formula (III) with a sulfonium salt of formula (I) in which X is methyl and Y is n-propyl and n-butyl.

The sulfonium salt of formula (I) is normally generated in situ, i.e. during the process, through the reaction with dimethyl sulfate of a dialkyl sulfide of formula (V),

X—S—Y (V),

where X and Y are as defined in formula (I). This is effected for example at from 50 to 150° C. In this reaction, the dialkyl sulfide of formula (V) is normally used in a slight molar excess, for example of from 1.01 to 1.2 mol, preferably from 1.01 to 1.15 mol, of dialkyl sulfide based on 1.00 mol of dimethyl sulfate. Since this reaction is exothermic, the temperature of the initially charged dialkyl sulfide of formula (I) and/or of the reaction mixture may also be below 50° C. at the start of the reaction. The addition of dimethyl sulfate increases the temperature of the reaction mixture even without additional heat being supplied.

The sulfonium salt thus obtained can either be used directly in the process according to the invention or else stored. The sulfonium salt of formula (II) is also normally generated in situ, i.e. during the process, through the reaction of a dialkyl sulfide of formula (V), in which X and Y are as defined in formula (I) and Z is chloride, bromide or iodide, with a methyl halide, for example methyl chloride, methyl bromide or methyl iodide. The dialkyl sulfide is normally used in a slight molar excess, for example of from 1.01 to 1.2 mol, preferably from 1.01 to 1.15 mol, of dialkyl sulfide based on 1.00 mol of methyl halide.

The sulfonium salt of formula (II), in which X and Y are as defined in formula (I) and Z is carbonate, is also normally generated in situ, i.e. during the process, through the reaction of a dialkyl sulfide of formula (V) with dimethyl carbonate. The dialkyl sulfide is normally used in a slight molar excess, for example of from 1.01 to 1.2 mol, preferably from 1.01 to 1.15 mol, of dialkyl sulfide based on 1.00 mol of dimethyl carbonate.

The sulfonium salts of formula (I) or of formula (II) are preferably prepared in a separate reactor, preferably the first reactor (A), particularly preferably in the amount required for the respective reaction of the aldehyde or ketone, preferably of the compound of formula (III).

In a preferred embodiment of the process according to the invention, the sulfonium salt of formula (I) or of formula (II) is therefore used in the form of a melt or of a solution.

In the process according to the invention, the reaction of the ketone or aldehyde, preferably of the compound of formula (III), can be effected without solvent or in the presence of at least one solvent. The process according to the invention is preferably carried out at least in the presence of the dialkyl sulfide of formula (V) as solvent.

0.1 to 3 molar equivalents, preferably from 1.1 to 1.5 molar equivalents, of dialkyl sulfide of formula (V) are normally used as solvent, based on the ketone or the aldehyde, preferably based on the compound of formula (III).

However, the process according to the invention can also be carried out in the presence of other solvents, for example toluene, xylene, chlorobenzene, water, preferably xylene, mixtures thereof or else mixtures of these solvents with dialkyl sulfide of formula (V).

The process according to the invention is carried out in the presence of a base, preferably in the presence of alkali metal hydroxide, more preferably in the presence of sodium hydroxide or potassium hydroxide. Particular preference is given to using potassium hydroxide as base. This can be for example potassium hydroxide in solid form, preferably in the form of flakes, powder, or a solution, particularly preferably in the form of flakes. Normally 1.5 to 2.0 molar equivalents of base, preferably alkali metal hydroxide, more preferably sodium hydroxide or preferably potassium hydroxide, based on the aldehyde or the ketone, preferably based on the compound of formula (III), are used.

In a preferred embodiment of the process according to the invention, from 1.0 to 2 mol, preferably from 1.0 to 1.3 mol, of sulfonium salt of formula (I) or (II) are used per mole of ketone or aldehyde, preferably per mole of compound of formula (III).

In the process according to the invention, the reaction is normally carried out at temperatures of from 20 to 100° C., preferably from 30 to 60° C.

The reaction of the aldehyde or ketone, preferably of the compound of formula (III), with the sulfonium salt of formula (I) or of formula (II) preferably takes place in a second reactor (B). In one embodiment of the process according to the invention in which the reaction of the aldehyde or ketone, preferably of the compound of formula (III), is carried out in the presence of solvent, the solvent, preferably the dialkyl sulfide of formula (V), is initially charged at the start of the process according to the invention. This may be followed by the addition of from 0.05 to 0.2 moles of water per mole of aldehyde or ketone, preferably per mole of compound of formula (III). This can promote the initiation of the exothermic reaction of the sulfonium salt of formula (I) or of formula (II) with the aldehyde or ketone, preferably with the compound of formula (III). If too much water is added, this in turn has an adverse effect on the initiation of the exothermic reaction.

The alkali metal hydroxide, preferably potassium hydroxide or sodium hydroxide, is then added to the reaction mixture, preferably to the reaction mixture in the second reactor (B). The reaction mixture, preferably the reaction mixture in the second reactor (B), is then heated to a temperature of from 30 to 50° C., preferably from 35 to 45° C. The reactor, preferably the second reactor (B), is then preferably inertized with an inert gas, for example nitrogen. This is followed by the addition of the sulfonium salt of formula (I) or of formula (II), preferably from the first reactor (A), to the reaction mixture, preferably to the reaction mixture in the second reactor (B). The addition normally takes place within 0.5 to 3 hours, preferably within 0.75 to 2 hours. During the addition, the temperature of the reaction mixture is held within a range from 30 to 50° C., preferably from 35 to 45° C. The reaction mixture, preferably the reaction mixture in the second reactor (B), is at the end of the addition of the amount of sulfonium salt of formula (I) or of formula (II) normally held for 1 to 4 hours at a reaction mixture temperature of from 30 to 50° C., preferably from 35 to 45° C. During and after the addition of the sulfonium salt of formula (I) or of formula (II), the reaction mixture is preferably mixed mechanically or hydraulically so as to obtain a mixture that is as homogeneous as possible.

The progress of the reaction can be determined by analyzing samples that have been worked up in the same way as the reaction mixture. The content of reactant and product can normally be determined by HPLC or gas chromatography, either as a percentage by area without external standard or as a percentage by weight with external standard.

Once the reaction has ended, the reaction mixture is preferably hydrolyzed with water. In one embodiment this may, preferably in a third reactor (C), be done by mixing with the reaction mixture, preferably with the reaction mixture in the second reactor (B), 1 to 2 kg, preferably 1.2 to 1.5 kg, of water, based on 1 kg of aldehyde or ketone used, preferably based on 1 kg of compound of formula (III) used. Mixing is preferably effected through mechanical and/or hydraulic mixing. Particular preference is given to initially charging the water, preferably in the third reactor (C), and adding the reaction mixture, preferably from the second reactor (B). To this mixture is then added for example 0.6 to 0.7 mol of hydrogen chloride, preferably in the form of 20 to 35% aqueous hydrochloric acid, per mole of aldehyde or ketone used, preferably per mole of compound of formula (III) used, so as to adjust the pH of the reaction mixture to 6 to 8, preferably to from 6.3 to 7.0. This hydrolysis step normally takes place at a reaction mixture temperature of from 20 to 60° C. Once mixing is complete, the hydrolyzed reaction mixture normally separates into an upper organic phase and a lower aqueous phase. The lower aqueous phase can then be run off, preferably from the second reactor (B), and the upper organic phase, preferably from the second reactor (B), then transferred to a further reactor, preferably to the third reactor (C). Low-boiling fractions of the reaction mixture can then be removed from the upper organic phase by distillation. This preferably takes place at a pressure of from 5 to 20 hPa, more preferably from 7 to 12 hPa. In a preferred embodiment, a low-boiling fraction is first distilled off, for example up to a bottoms temperature of 91° C. at 10 hPa. n-Butyl methyl sulfide is then distilled off as a second fraction, for example up to a bottoms temperature of 91° C. at 10 hPa. This recovered n-butyl methyl sulfide can be reused in a subsequent reaction of the same type.

The fraction left behind as the distillation bottoms contains the epoxide as the reaction product, preferably the epoxide of formula (IV), normally having a content of more than 85% by weight, preferably having a content of at least 90% by weight, in yields of 93 to 100% of theory. The product can normally be used as a reactant in a chemical reaction without further purification or processing. The epoxide of formula (IV) is normally used in an epoxide opening with a triazole to give cyproconazole.

Starting from an aldehyde or ketone, preferably from the compound of formula (III), it is surprisingly possible, using the process according to the invention, to obtain epoxides, preferably the compound of formula (IV), in high purities and yields.

The invention additionally relates to the sulfonium salts of formula (I) and of formula (II) that are used in the process according to the invention.

The invention further relates to the use of the sulfonium salts of formula (I) and of formula (II) in the process according to the invention for the preparation of epoxides.

### EXAMPLES

### 2-(4-Chlorophenyl)-2-(1-cyclopropylethyl)oxirane

### a) Preparation of the sulfonium Salt butylmethylsulfonium methosulfate

A first reactor (A) was charged with 1800 g (16.93 mol) of n-butyl methyl sulfide and this was then heated to 40° C. To this was then metered in with stirring 1903 g (14.94 mol) of dimethyl sulfate and the temperature of the reaction mixture was increased to 100° C. over the course of 1 hour. The reaction mixture was left at 100° C. for 1 hour with stirring and then cooled to 40° C. over the course of 1 hour.

### b) Epoxidation

A second reactor (B) was charged with 1300 g (12.23 mol) of n-butyl methyl sulfide. To the n-butyl methyl sulfide in the second reactor (B) was then added 26 g of water (1.43 mol) and 1412 g (21.39 mol) of potassium hydroxide and the temperature of the mixture in the second reactor (B) was increased to 40° C. The second reactor (B) was then inertized with nitrogen. To the mixture in the second reactor (B) was then added 3000 g (content: 92.2% by weight, 13.23 mol) of 1-(4-chlorophenyl)-2-cyclopropylpropan-1-one [compound of formula (III)]. The contents of the first reactor (A), which contains the sulfonium salt in the form of a melt, were metered into the mixture in the second reactor (B) at 40° C. over the course of 120 minutes while stirring vigorously. The first reactor (A) was rinsed with 500 g of n-butyl methyl sulfide. The rinse solution from the first reactor (A) was then metered into the second reactor (B), likewise at 40° C., with vigorous stirring. The reaction mixture was then stirred at 40° C. for two hours. Once the reaction had ended, the reaction mixture was hydrolyzed. This was done by charging a third reactor (C) with 4000 g of water having a temperature of 25° C. and adding the reaction mixture from the second reactor (B) to the third reactor (C) while stirring. The pH of the reaction mixture was then at 25° C. adjusted to pH 6-7 with 1060 g (8.72 mol) of 30% hydrochloric acid while stirring vigorously. The mixture was then left to stand to allow phase separation, resulting in a two-phase mixture formed from an upper organic phase and a lower aqueous phase. The lower aqueous phase was run off and the upper organic phase then washed with 4000 g of water. A low-boiling fraction was then first distilled off from the washed organic phase by distillation at 10 hPa up to a bottoms temperature of not more than 91° C. 3774 g of n-butyl methyl sulfide (content: at least 90% by weight) was then distilled off at 91° C. and 10 hPa, leaving behind in the bottoms 3157 g of 2-(4-chlorophenyl)-2-(1-cyclopropylethyl)oxirane [compound of formula (IV)] (content: 90% by weight, yield: 96.6% of theory). The recovered n-butyl methyl sulfide can be reused in a subsequent reaction for the preparation of 2-(4-chlorophenyI)-2-(1-cyclopropylethyl)oxirane.

1-13. (canceled)

14. A process for preparing epoxides, in which a ketone or aldehyde is reacted at least with a sulfonium salt of formula (I), 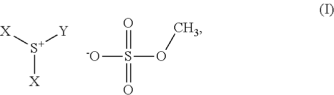
and/or a sulfonium salt of formula (II),
X₂SY+Z⁻ (II),
where in formula (I) and formula (II) X is methyl and Y is linear C₂ to C₁₁ alkyl,
and where in formula (II) Z is chloride, bromide, iodide or carbonate,
in the presence of a base.

15. The process according to claim 14, wherein in formula (I) and formula (II) X is ethyl, n-propyl, n-butyl, n-pentyl, n-heptyl, n-octyl, n-nonyl, n-decyl or n-undecyl.

16. The process according to claim 14, wherein said base is alkali metal hydroxide, more preferably sodium hydroxide or potassium hydroxide.

17. The process according to claim 14, wherein the ketone is the compound of formula (III), 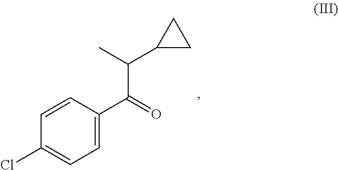
and the epoxide is the compound of formula (IV),

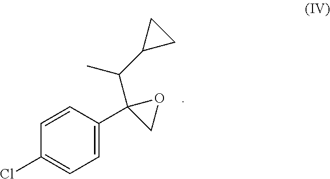

18. The process according to claim 14, wherein the sulfonium salt of formula (I) or (II) is used in the form of a melt or of a solution.

19. The process according to claim 14, wherein the reaction is effected without solvent or in the presence of at least one solvent.

20. The process according to claim 14, wherein at least one solvent is a dialkyl sulfide of formula (V),
X—S—Y (V),
where X and Y are as defined in formula (I).

21. The process according to claim 14, wherein the aldehyde or ketone is reacted in the presence of from 0.1 to 3 molar equivalents of dialkyl sulfide of formula (V) as solvent, based on the aldehyde or ketone.

22. The process according to claim 17, wherein the compound of formula (III) is reacted in the presence of from 1.1 to 1.5 molar equivalents of dialkyl sulfide of formula (V) as solvent, based on the compound of formula (III).

23. The process according to claim 14, wherein the aldehyde or ketone is reacted with from 1.0 to 2 molar equivalents of sulfonium salt of formula (I) or (II), based on the aldehyde or ketone.

24. The process according to claim 17, wherein the compound of formula (III) is reacted with from 1.0 to 1.3 molar equivalents of sulfonium salt of formula (I) or (II), based on the compound of formula (III).

25. The process according to claim 14, wherein the reaction is carried out at temperatures of from 20° C. to 100° C.

26. The process according to claim 14, wherein the reaction is carried out at temperatures of from 30° C. to 60° C.

27. The process according to claim 14, wherein the sulfonium salt of formula (I) is obtained by reacting a dialkyl sulfide of formula (V) with dimethyl sulfate.

28. The process according to claim 14, wherein the sulfonium salt of formula (II), in which X and Y are as defined in formula (II) and Z is chloride, bromide or iodide, is obtained by reacting a dialkyl sulfide of formula (V) with methyl chloride, methyl bromide or methyl iodide, depending on the radical Z.

29. The process according to claim 14, wherein the sulfonium salt of formula (II), in which X and Y are as defined in formula (I) and Z is carbonate, is obtained by reacting a dialkyl sulfide of formula (V) with dimethyl carbonate.

30. A sulfonium salt of formula (I) and of formula (II) according to claim 14.
